(19) United States
(12) Patent Application Publication
(10) Pub. No.: US 2025/0366913 A1
(43) Pub. Date: Dec. 4, 2025

(54) PERFORATION ASSEMBLY WITH CONDUCTIVE ELECTRODE

(71) Applicant: EDWARDS LIFESCIENCES CORPORATION, Irvine, CA (US)

(72) Inventors: Peleg Harel, Haifa (IL); Eitan Atias, Tel Aviv (IL); Noa Axelrod Manela, Kadima (IL)

(73) Assignee: EDWARDS LIFESCIENCES CORPORATION, Irvine, CA (US)

(21) Appl. No.: 19/302,467

(22) Filed: Aug. 18, 2025

Related U.S. Application Data
(63) Continuation of application No. PCT/US2024/017661, filed on Feb. 28, 2024.
(60) Provisional application No. 63/449,560, filed on Mar. 2, 2023.

(51) Int. Cl. A61B 18/14; A61B 18/00; A61F 2/24

(52) U.S. Cl. CPC A61B 18/1492; A61F 2/2427; A61B 2018/00369; A61B 2018/00601

(57) ABSTRACT

The present disclosure relates to that can be used for forming an opening in a target tissue, such as a host leaflet within which a guest prosthetic valve can be expanded. In an example, a perforation assembly includes a delivery apparatus and an electrode, wherein a distal portion of the electrode, comprising a non-straight shape in a free state thereof, is configured to extend distally from the delivery apparatus.

### CROSS-REFERENCE TO RELATED APPLICATIONS

This application is a continuation of International Application No. PCT/US2024/017661, filed Feb. 28, 2024, which claims the benefit of U.S. Provisional Application No. 63/449,560, filed Mar. 2, 2023, which is incorporated by reference herein.

### FIELD

The present disclosure relates to perforation tools that can be used to form an opening in a target tissue, and to methods and devices for cutting through a target tissue that can be a leaflet of an existing valvular structures (for example, leaflets or commissures of a native heart valve or previously-implanted prosthetic valve) prior to or during implantation of a prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

One known technique for mitigating the risk of coronary ostial obstruction involves lacerating or severing a portion of one or more leaflets of the host valve (which can be an aortic bioprosthetic valve or a native aortic valve). Lacerating or severing a portion of the leaflet(s) reduces the risk of blocking the coronary ostia when the guest prosthetic valve is implanted and displaces the leaflets of the host valve toward the inner wall of the aortic root. However, methods that rely on lacerating existing leaflets, require high spatial precision and surgical skill. Moreover, once the leaflets have been lacerated, the existing heart valve may function poorly and increase the risk of aortic insufficiency, at least until a replacement prosthetic valve comprises been successfully implanted. If the existing leaflets have become calcified, there is a further risk that the lacerating will release particulate or other debris into the blood stream, which may make the patient susceptible to vascular occlusion or stroke. In some examples, a perforation assembly is provided, the perforation assembly comprising: a delivery apparatus; and an electrode extending from a proximal portion to a distal portion thereof, through the delivery apparatus, the distal portion of the electrode configured to extend distally from the delivery apparatus.

In some examples, the distal portion of the electrode comprises a non-straight shape in a free state thereof.

In some examples, a perforation method is provided, the method comprising: distally extending a distal portion of an electrode from a delivery apparatus to a predetermined anatomical location; and providing an electric current to the electrode such that the electric current creates a perforation in a section of material at the predetermined anatomical location.

In some examples, the electrode comprises a non-straight shape in a free state thereof.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a cross-sectional view of a native aortic valve.

FIG. 2A shows a cross-sectional view of a prosthetic heart valve implanted in the native aortic valve of FIG. 1, according to some examples of the present disclosure.

FIG. 2B shows the implanted prosthetic heart valve of FIG. 1A as viewed from the ascending aorta, according to some examples of the present disclosure.

FIG. 3 shows a valve-in-valve implantation within the native aortic valve of FIG. 1, according to some examples of the present disclosure.

FIG. 4 shows an exemplary perforation assembly, according to some examples of the present disclosure.

FIG. 5A shows a first example of an electrode of the exemplary perforation assembly of FIG. 4, in a contained state, according to some examples of the present disclosure.

FIGS. 5B-5C show the electrode of FIG. 5A in a free state, according to some examples of the present disclosure.

FIG. 6A shows a cut-away view of a distal portion of the perforation assembly of FIG. 4, with the electrode of FIGS. 5A-5C retracted, according to some examples of the present disclosure.

FIG. 6B shows a cut-away view of a distal portion of the perforation assembly of FIG. 4, with the electrode of FIGS. 5A-5C distally advanced, according to some examples of the present disclosure.

FIG. 6C shows a side view of the distal portion of the perforation assembly of FIG. 6B.

FIG. 7A shows a second example of an electrode of the exemplary perforation assembly of FIG. 4, in a contained state, according to some examples of the present disclosure.

FIG. 7B shows the electrode of FIG. 7A in a free state, according to some examples of the present disclosure.

FIG. 8A shows a third example of an electrode of the exemplary perforation assembly of FIG. 4, in a contained state, according to some examples of the present disclosure.

FIG. 8B shows the electrode of FIG. 8A in a free state, according to some examples of the present disclosure.

FIGS. 9A-9D show various steps of a perforation method, according to some examples of the present disclosure.

FIGS. 10A-10B show various outcomes of the method of FIGS. 9A-9D when using the electrode of FIGS. 8A-8B.

FIG. 11 shows a perspective view an exemplary delivery assembly, according to some examples of the disclosure.

FIGS. 12A-12D show various steps of a method utilizing the delivery assembly of FIGS. 8-9, according to some examples of the disclosure.

FIG. 13 shows a previously implanted prosthetic valve subsequent to forming a leaflet opening in a host leaflet, according to some examples of the disclosure.

FIG. 14 shows a configuration in which a second prosthetic valve comprises been expanded within the leaflet opening of a host prosthetic valve, according to some examples of the disclosure.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular example and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In some examples, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first,” “second,” and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a,” “b,” “c,” etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting prosthetic valves and modifying leaflets of an existing valvular structure in a patient's heart. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve comprises been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some examples, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

While implanting a guest valve within a host valve in both native TAVR and ViV TAVR, there is a risk of the guest valve displacing the leaflets of the host valve (either the native leaflets or the leaflets of a previously implanted prosthetic valve) outwards towards the coronary ostia. In some cases, the displacement can be such that a host leaflet partially or fully blocks a coronary ostium, causing coronary artery obstruction. The risk of coronary artery obstruction can be relatively higher in ViV TAVR compared to native TAVR. In some examples, some of the below devices and methods can mitigate such an issue.

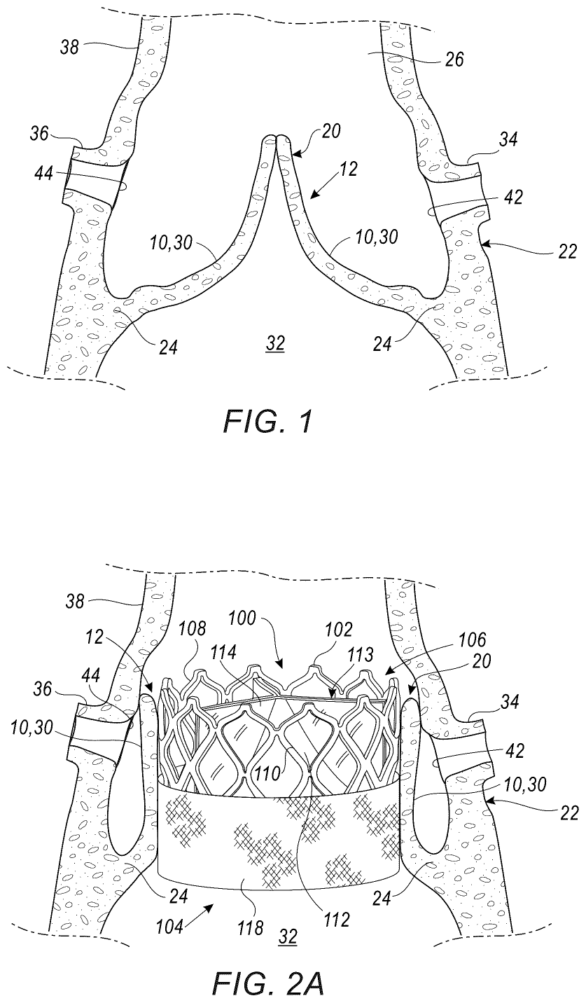

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 comprises three leaflets (only two leaflets are visible in the simplified illustration of FIG. 1), but aortic valves with fewer than three leaflets are possible. The leaflets 30 are supported at native commissures 40 (see FIG. 1B) by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

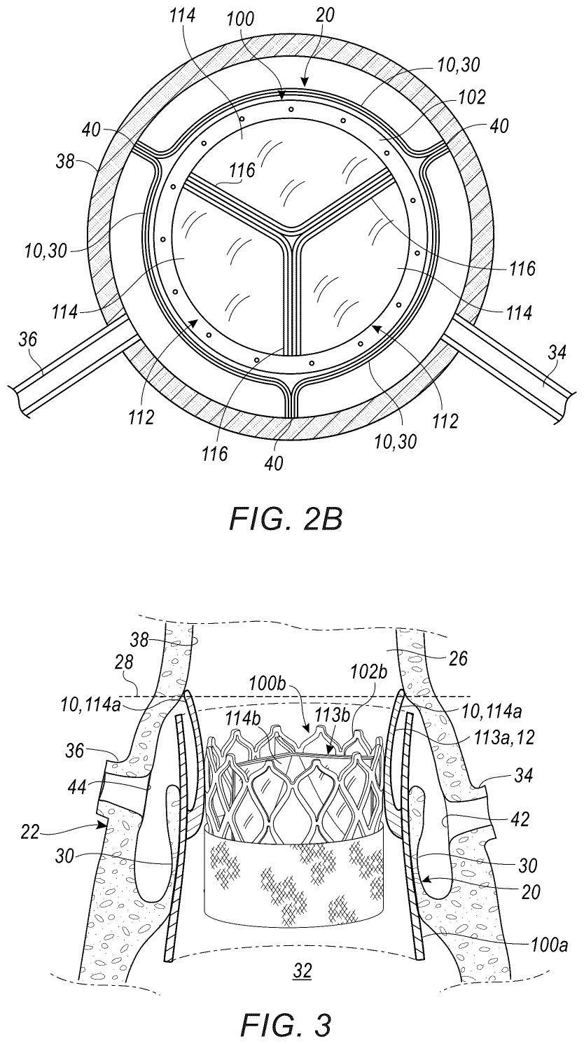

FIGS. 2A-2B show an exemplary prosthetic valve 100 that can be implanted in a native heart valve, such as the native aortic valve 20 of FIG. 1. FIG. 2A shows a side view of prosthetic valve 100 and FIG. 2B show a top view of prosthetic valve 100.

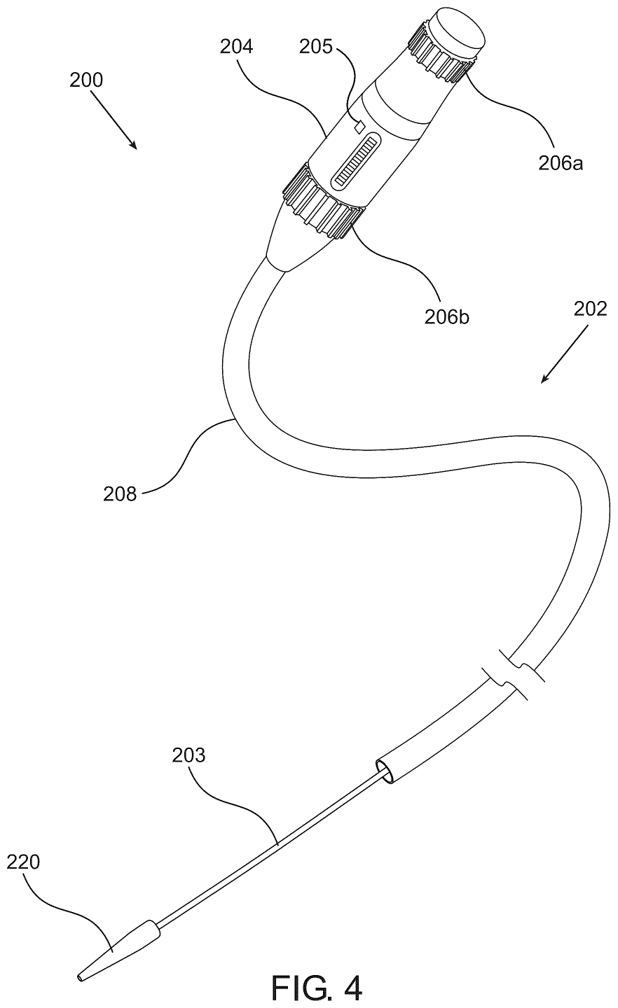

The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (such as delivery apparatus 202 described below with respect to FIG. 4, as well as other examples of delivery apparatuses described throughout the current disclosure) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

As described above, FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the distal end of the proximal valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can be made of various suitable materials, including plastically-deformable materials such as, but not limited to, stainless steel, a nickel based alloy (for example, a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 is an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can include a plurality of angled struts and vertical or axial struts. At least some of the struts 108 can be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 124 can be arranged together to form prosthetic valve commissures 116 that are coupled (directly or indirectly) to respective portions of the frame 102, thereby securing at least a portion of the valvular structure 113 to the frame 102. The leaflets 124 can be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can comprise at least one skirt or scaling member. For example, the prosthetic valve 100 can include an inner skirt (not shown in FIG. 2A-2B), which can be secured to the inner face of the frame 102. Such an inner skirt can be configured to function, for example, as a scaling member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner face of frame 102, while the leaflets can be sutured to the inner skirt along a scalloped line (not shown). An inner skirt can be coupled to the frame 102 via sutures or another form of coupler.

The prosthetic valve 100 can comprise, in some examples, an outer skirt 118 mounted on the outer face of frame 102 (as shown in FIGS. 2A-2B), configured to function, for example, as a scaling member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can be formed of a single sheet of material that extends continuously around the inner face of frame 102. In some cases, the outer skirt 118 can be formed of a single sheet of material that extends continuously around the outer face of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the valve 100 is at the STJ level 28 and the diameter of the valve 100 is similar to the STJ diameter such that the frame 102 touches or is very close to the aortic wall 38 at the STJ level 28), the host leaflets 10 may compromise the ability for future access into the coronary arteries 34, 36 or perfusion through the valve frame 102 to the coronary arteries 34, 36 during the diastole phase of the cardiac cycle. Similar problems may occur in some patient anatomies cither when a prosthetic guest valve 100b is percutaneously expanded within a previously implanted prosthetic host valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can be a surgically implanted prosthetic heart valve (known as a “surgical valve”) or a transcatheter heart valve. The term “guest valve”, as used herein, refers to a prosthetic valve implanted in a host valve, which can be either a native heart valve or a previously implanted prosthetic valve. Similarly, the term “host leaflets 10”, as used herein, refers to native leaflets 30 of a native valve in which a new prosthetic guest valve 100 is implanted, or to prosthetic valve leaflets 114a of a previously implanted host valve 100a in which a new prosthetic guest valve 100b is implanted.

To avoid obstruction of blood flow to the coronary arteries 34, 36, the valvular structure of the existing host valve (whether a native aortic valve or a previously implanted prosthetic valve) can be modified by components of a delivery apparatus prior to or during implantation of a new prosthetic valve within the existing valvular structure. In some examples, the host valvular structure 12 is modified by piercing, lacerating, tearing, slicing, and/or cutting one or more host leaflets 10 (for example, a free end of the host leaflet 10 or a commissure of host adjacent leaflets 10, which can be a native commissure 40 for a native aortic host valve 20, or a prosthetic valve commissure 116 for a previously implanted prosthetic host valve 100) using the delivery apparatus. The modification thus disrupts the impermeable tubular structure that would otherwise be formed by the existing host leaflets 10, thereby allowing blood to flow to the coronary arteries 34, 36. Any delivery apparatus described throughout the current disclosure, is advantageously configured to modify the host valvular structure 12 (i.e., modify at least one of the host leaflets 10), and implant a guest prosthetic valve 100 within the modified valvular structure, without the need to switch between separate delivery apparatuses for each function.

Any delivery assembly disclosed herein, comprises a delivery apparatus according to any of the examples described below, and a balloon expandable prosthetic valve. While examples of a delivery assembly described in the current disclosure, are shown to include an exemplary delivery apparatus and a balloon expandable prosthetic valve, it should be understood that a delivery apparatus according to any example of the current disclosure can be used for implantation of other prosthetic devices aside from prosthetic valves, such as stents or grafts.

A delivery assembly comprising any delivery apparatus described throughout the current disclosure can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

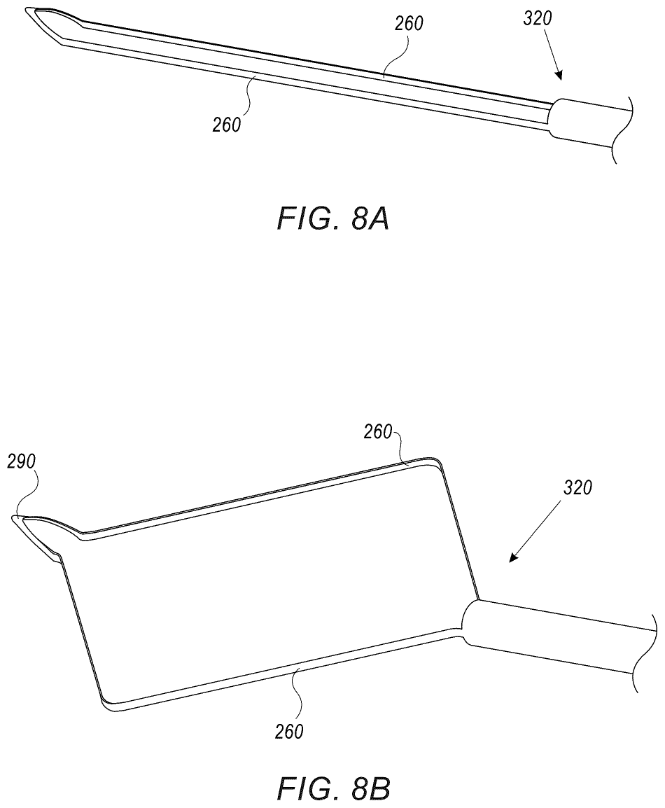

FIG. 4 illustrates an exemplary perforation assembly 200. According to some examples, perforation assembly comprises a delivery apparatus 202. According to some examples (as shown and described further below in relation to FIG. 8), delivery apparatus 202 is adapted to deliver a prosthetic valve, such as prosthetic valve 100 described above with respect to FIGS. 2A-2B. According to some examples, the delivery apparatus 202 comprises a handle 204 and a nosecone shaft 203 extending distally from handle 204. According to some examples, the delivery apparatus 202 further comprises a nosecone 220, the proximal end of the nosecone shaft 203 secured to the handle 204 and the nosecone 220 secured to the distal end of the nosecone shaft 203. According to some examples, an outer delivery shaft 208 can concentrically extend over the nosecone shaft 203. According to some examples (as shown and described below in relation to FIG. 8), the delivery apparatus 202 comprises an inflatable balloon mounted on its distal end.

According to some examples, the delivery apparatus 202 comprises an electric current source 205 configured to generate and output a predetermined electric current. According to some examples, the electric current source 205 is secured within handle 204. Alternatively, the electric current source 205 can be external to handle 204.

According to some examples, the delivery apparatus 202 further comprises an electrode, as will be described below in relation to electrodes 250, 300 and 320, the electrode receiving power from the electric current source 205. As will be described below, electrodes 250, 300 and 320 are configured to act as a perforation mechanism, also described herein, as a means for creating a perforation. According to some examples, the delivery apparatus 202 further comprises a guidewire 230 extending through the nosecone shaft 203 and the nosecone 220.

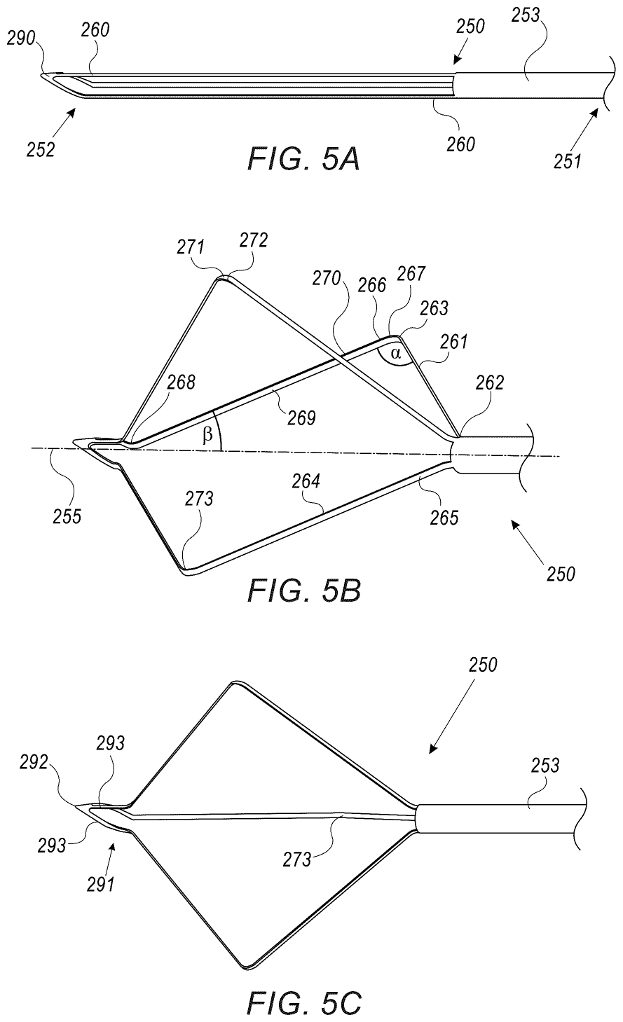

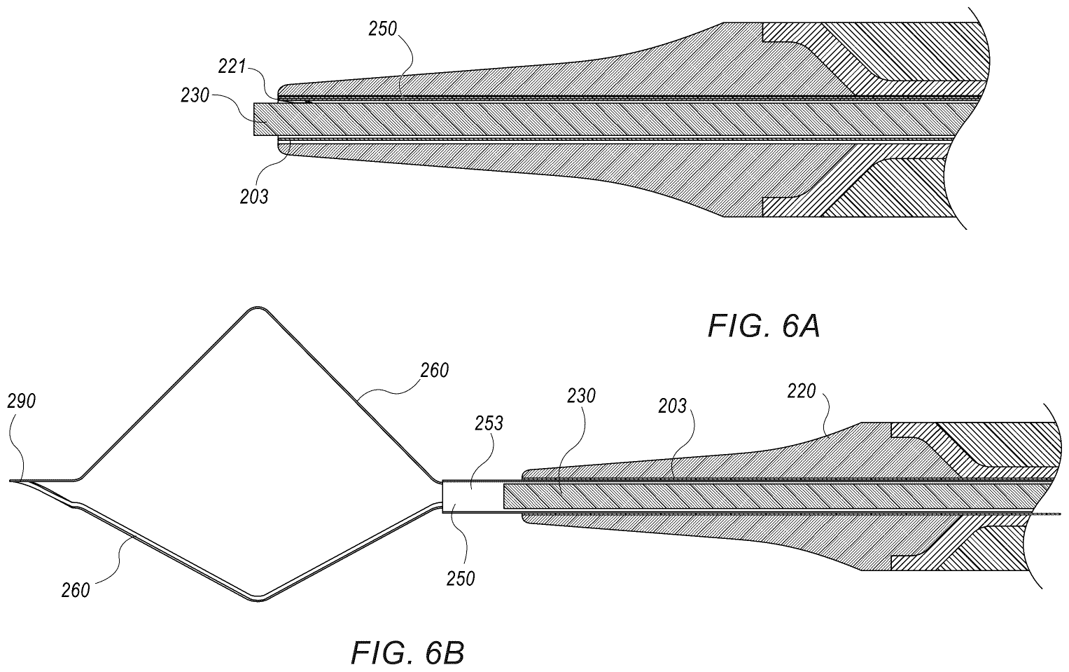

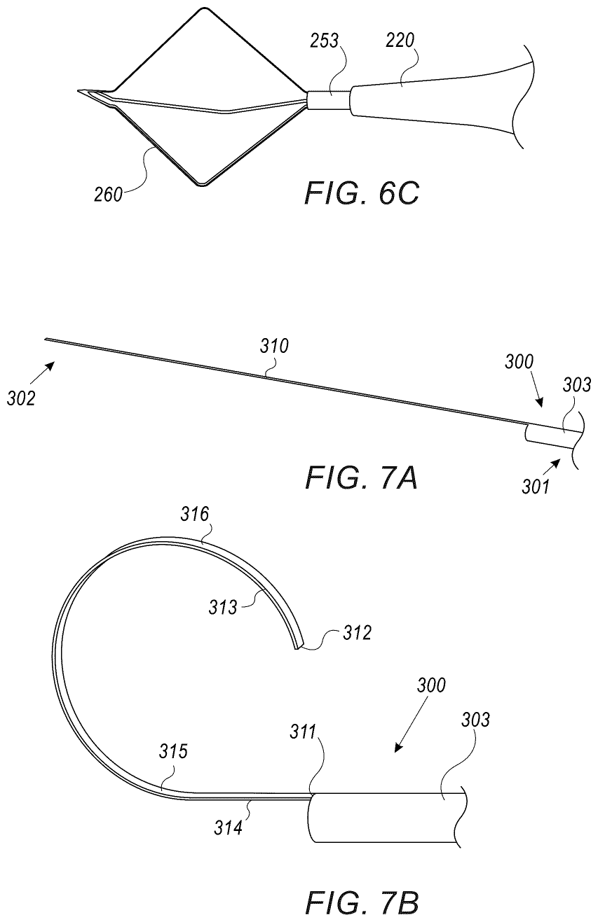

According to some examples, the delivery apparatus 202 comprises an electrode 250. FIG. 5A shows electrode 250 in a contained state and FIG. 5B shows electrode 250 in a free state, as will be described below. FIG. 6A shows a cut-away view of a distal section of the delivery apparatus 202, comprising electrode 250 enclosed within the nosecone 220. FIG. 6B shows a cut-away view of a distal section of the delivery apparatus 202, comprising electrode 250 in a free state, when distally extended out of the nosecone 220. FIG. 6C shows a perspective view of distal section of the delivery apparatus 202 shown in FIG. 6B.

The electrode 250 extends from a proximal portion 251 to a distal portion 252. According to some examples, proximal portion 251 or electrode 250 extends proximally towards handle 204, optionally enclosed by outer delivery shaft 208. According to some examples, proximal portion 251 of electrode 250 reaches handle 204. According to some examples, as will be described below, distal portion 252 of the electrode 250 is configured to extend distally from the delivery apparatus 202.

According to some examples, the electrode 250 further comprises a center portion 253 extending between the proximal portion 251 and the distal portion 252 thereof. According to some examples, the cross-section of the center portion 253 of the electrode 250 comprises a generally closed shape. The term “closed shape”, as used herein, means a shape that forms an enclosure. According to some examples, the center portion 253 of the electrode 250 is generally tubular shaped. According to some examples, the guidewire 230 extends through the center portion 253 of the electrode 250 and is enclosed by the closed shape thereof.

According to some examples, as will be described below, the distal portion 252 of the electrode 250 comprises a non-straight shape when in a free state. The term “non-straight shape”, as used herein, means a shape that at least a portion thereof is not parallel to a longitudinal axis 255 defined by the electrode 250. The term “free state”, as used herein, means that it is not constrained by a mechanical structure.

According to some examples, as will be described below, the distal portion 252 of the electrode 250 is configured to extend distally from the delivery apparatus 202. According to some examples, when the distal portion 252 of the electrode 250 extends distally from the delivery apparatus 202, it is not constrained by the nosecone 220 and is in the free state. According to some examples, the electrode 250 extends through the nosecone shaft 203 and the nosecone 220, and the distal portion 252 of the electrode 250 is configured to extend distally out of the nosecone 220 via an opening 221 at the distal end of the nosecone 220.

According to some examples the distal portion 252 of the electrode 250 is pre-shaped (as known to those skilled in the art) such that responsive to the distal portion 252 of the electrode 250 being released from an enclosure the distal portion 252 forms into the non-straight shape, as described above. For example, the distal portion 252 of the electrode 250 can initially be secured within the nosecone 220, with the distal portion 252 being straight due to the structural confinement of the nosecone 220. Thereafter, when the electrode 250 is advanced distally, the distal portion 252 exits the nosecone 220 through the opening 221 and assumes the non-straight shape. Thus, in the free state thereof, the electrode 250 or electrode distal portion 252 is in a state in which the distal portion 252 is not contained in an enclosure that prevents it from assuming its non-straight shape, such as a channel axially extending through the nosecone 220 or a lumen of the nosccone shaft 203.

According to some examples, the electrode 250 consists essentially of one or more electrically conductive materials, such as stainless steel and/or Nickel Titanium (Nitinol).

According to some examples, the handle 204 comprises a steering mechanism configured to adjust the curvature of the distal end portion of the delivery apparatus 202. In the illustrated example, the handle 204 includes an adjustment member, such as the illustrated rotatable knob 206a, which in turn is operatively coupled to the proximal end portion of a pull wire. The pull wire can extend distally from the handle 204 through the outer delivery shaft 208 and comprises a distal end portion affixed to the outer delivery shaft 208 at or near the distal end of the outer delivery shaft 208. Rotating the knob 206a can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein. The adjustment member can thus be defined as a means for adjusting the curvature of the distal end portion of the delivery apparatus 202.

According to some examples, the handle 204 further comprises an adjustment mechanism, such as the illustrated rotatable knob 206b. The adjustment mechanism 206b can be configured to advance the electrode 250 distally from the nosccone 220 to a desired location. According to some examples, by distally advancing the electrode 250, the amount of the distal portion 252 that is exposed can be adjusted. The adjustment mechanism can thus be defined as a means for advancing the electrode 250.

Additionally, the adjustment mechanism 206b can retract the electrode 250 proximally through the nosecone shaft 203. The adjustment mechanism can thus further be defined as a means for retracting the electrode 250. Although the advancement mechanism is show herein as a rotatable knob 206b, this is not meant to be limiting in any way, and any suitable mechanism for advancing the electrode 250 can be provided, as known to those skilled in the art. According to some examples, the adjustment mechanism is configured to rotate the electrode 250 about itself. According to some examples (not shown), an additional adjustment mechanism can be provided to distally (and optionally proximally) advance the guidewire 230.

Although the above comprises been described in relation to a combination of elements, this is not meant to be limiting in any way. In some examples, the electrode 250 can be provided without a prosthetic valve 100, without an inflatable balloon, without a nosccone 220, without a nosccone shaft 203, without a guidewire 230 and/or without a handle 204.

According to some examples, the distal portion 252 of the electrode 250 comprises at least one strut 260. Although the electrode 250 is illustrated and described herein as comprising three struts 260, this is not meant to be limiting in any way. In some examples, the electrode 250 can comprise 1, 2, 3, or more than 3 struts.

According to some examples, when in a contained state, struts 260 are generally straight. The term “contained state”, as used herein, means a state where struts 260 are mechanically contained by a structure at least partially surrounding struts 260. In some examples, the surrounding structure completely surrounds a cross-section of struts 260. According to some examples, when in the free state, each of the struts 260 comprises: a rear section 261; and a forward section 266. Rear section 261 extends from a proximal end 262 to a distal end 263. Rear section 261 further comprises an inner face 264 and an outer face 265, outer face 265 opposing the inner face 264. Forward section 266 extends from a proximal end 267 to a distal end 268. Forward section 266 further comprises an inner face 269 and an outer face 270. According to some examples, for each strut 260, inner faces 264 and 269 generally face longitudinal axis 255 of the electrode 250.

According to some examples, for each strut 260, the forward section 266 is an extension of the rear section 261. Thus, in such examples, no additional parts, or gaps, are present between rear section 261 and forward section 266.

According to some examples, for each strut 260, an angle α is defined between the inner face 264 of the rear section 261 and the inner face 269 of the forward section 266. According to some examples, for each strut 260, in the free state thereof, the angles α are substantially equal. According to some examples, for each strut 260, the angle α is a non-zero and a non-straight angle, i.e. angle α is greater than zero degrees and less than 180 degrees. According to some examples, for each strut 260, angle α is less than 180 degrees. According to some examples, for each strut 260, angle α is about 60-120 degrees.

According to some examples, for each strut 260, the rear section 261 extends away from the longitudinal axis 255 of the electrode 250 and the forward section 266 extends towards the longitudinal axis 255 of the electrode 250. Thus, when extending from the proximal end 262 to the distal end 263, the rear section 261 extends away from the longitudinal axis 255. Similarly, when extending from the proximal end 267 to the distal end 268, the forward section 266 extends towards the longitudinal axis 255. Thus, in such examples, angle α of each strut 260 faces longitudinal axis 255.

According to some examples, for each strut 260, a predetermined angle β is defined between the inner face 269 of the forward section 266 and the longitudinal axis 255 of the electrode 250. According to some examples, for each strut 260, in the free state thereof, the angles β are substantially equal. According to some examples, for each strut 260, the angle β is a non-zero and a non-straight angle, i.e. angle β is greater than zero degrees and less than 180 degrees. According to some examples, for each strut 260, angle β is less than, or equal to, 90 degrees. According to some examples, for each strut 260, angle β is about 20-70 degrees.

According to some examples, for each strut 260, the distal end 263 of the rear section 261 and the proximal end 262 of the forward section 266 are each generally curved. According to some examples, for each strut 260, the distal end 263 of the rear section 261 and the proximal end 262 of the forward section 266 define a center section 271 of the strut 260. The center section 271 comprises an outer face 272 and an inner face 273, inner face 273 opposing outer face 272 and facing longitudinal axis 255. According to some examples, the outer face 272 of the center section 271 comprises a generally convex shape and the inner face 273 of the center section 271 comprises a generally concave shape.

According to some examples, for each strut 260, the proximal end 262 of the rear section 261 is generally curved. According to some examples, for each strut 260, the inner face 264 of the proximal end 262 of the rear section 261 comprises a generally convex shape. Similarly, the outer face 265 of the proximal end 262 of the rear section 261 comprises a generally concave shape.

According to some examples, for each strut 260, the distal end 268 of the forward section 266 is generally curved. According to some examples, for each strut 260, the inner face 269 of the distal end 268 of the forward section 266 comprises a generally convex shape. Similarly, the outer face 270 of the distal end 268 of the forward section 266 comprises a generally concave shape.

Although struts 260 are illustrated and described herein as comprising a rear section 261 and a forward section 266, with an angle α therebetween, this is not meant to be limiting in any way. In some examples, forward sections 266 can be provided without rear sections 261, thereby presenting an angled strut 260. As will be described below, rear sections 261 allow struts 260 to be retracted back into the nosecone 220.

According to some examples, the distal portion 252 of the electrode 250 comprises a front section 290. Front section 290 extends distally from the distal end 268 of the forward section 266 of each strut 260. According to some examples, the front section 290 extends from a proximal end 291 to a distal end 292. According to some examples, the distal end 292 of the front section 290 forms a tip. According to some examples, the tip formed by the distal end 292 of the front section 290 defines a generally triangle shape.

According to some examples, the front section 290 of the distal portion 252 of the electrode 250 comprises a plurality of arms 293. According to some examples, each arm 293 extends from the distal end 268 of the forward section 266 of a respective strut 260. Thus, according to some examples, each arm 293 is an extension of a respective strut 260.

According to some examples, each strut 260 is attached to the center portion 253 of the electrode 250. According to some examples, the proximal end 262 of the rear section 261 of each strut 260 is attached to the center portion 253. According to some examples, each strut 260 extends from the center portion 253 of the electrode 250. According to some examples, the proximal end 262 of the rear section 261 of each strut 260 is extends from the center portion 253.

According to some examples, a distal portion of the guidewire 230 comprises an electrically conductive surface. According to some examples, the entirety of the outer surface of the distal portion of the guidewire 230 is electrically conductive. Alternatively, a portion of the outer surface of the distal portion of the guidewire 230 is electrically insulated.

According to some examples, the electric current source 205 generates an electric current, and outputs the generated electric current to the electrode 250. According to some examples, the electric current is further output to the guidewire 230. According to some examples, the generated electric current is an alternating current. According to some examples, the frequency of the alternating current is a radio-frequency (RF), i.e. from about 20 kHz to 300 GHz. According to some examples, the electrode 250 conducts electricity from the proximal portion 251 to the distal portion 252, via the center portion 253. According to some examples, the distal portion 252 of the electrode 250, and optionally the electrically conductive surface of the guidewire 230 is configured to output RF energy.

According to some examples, the electric current source 205 is configured, responsive to a user input, to alternately provide the electric current and not provide the electric current. For examples, the electric current source 205 comprises a user input device (not shown), such as a switch that can turn the electric current source on and off. Thus, as will be described below, the electric current can preferably be provided only when the distal portion 252 of the electrode 250 is in the correct location.

According to some examples, as described above, the delivery apparatus 202 comprises an electrode 300. FIG. 7A shows the electrode 300 in a contained state and FIG. 7B shows the electrode 300 in a free state. According to some examples, the electrode 300 extends from a proximal portion 301 to a distal portion 302. According to some examples, the electrode 300 further comprises a center portion 303 extending between the proximal portion 301 and the distal portion 302 thereof. According to some examples, as described above in relation to center portion 253 of electrode 250, the cross-section of the center portion 303 of the electrode 300 comprises a generally closed shape. According to some examples, the center portion 303 of the electrode 300 is generally tubular shaped. According to some examples, the guidewire 230 extends through the center portion 303 of the electrode 300 and is enclosed by the closed shape thereof.

According to some examples, as described above in relation to the distal portion 252 of the electrode 250, the distal portion 302 of the electrode 300 comprises a non-straight shape when in a free state. According to some examples, as described above in relation to electrode 250, the distal portion 302 of the electrode 300 is configured to extend distally from the delivery apparatus 202 (shown in FIG. 4). According to some examples, when the distal portion 302 of the electrode 300 extends distally from the delivery apparatus 202, it is not constrained by the nosecone 220 (shown in FIG. 4) and is in the free state. According to some examples, the electrode 300 extends through the nosecone shaft 203 (shown in FIG. 4) and the nosecone 220, and the distal portion 302 of the electrode 300 is configured to extend distally out of the nosecone 220 via an opening 221 at the distal end of the nosecone 220 (shown in FIG. 6A in relation to electrode 250).

According to some examples, as described above in relation to electrode 250, the distal portion 302 of the electrode 300 is pre-shaped (as known to those skilled in the art) such that responsive to the distal portion 302 of the electrode 300 being released from an enclosure the distal portion 302 forms into the non-straight shape, as described above. For example, the distal portion 302 of the electrode 300 can initially be secured within the nosecone 220, with the distal portion 302 being straight due to the structural confinement of the nosecone 220. Thereafter, when the electrode 300 is advanced distally, the distal portion 302 exits the nosecone 220 through the opening 221 and assumes the non-straight shape. Thus, in the free state thereof, the electrode 300 or electrode distal portion 302 is in a state in which the distal portion 302 is not contained in an enclosure that prevents it from assuming its non-straight shape, such as a channel axially extending through the nosecone 220 or a lumen of the nosecone shaft 203.

According to some examples, as described above in relation to the electrode 250, the electrode 300 consists essentially of one or more electrically conductive materials, such as stainless steel and/or Nickel Titanium (Nitinol). According to some examples, as described above in relation to the electrode 250, a steering mechanism of the handle 204 (shown in FIG. 4) is configured to distally advance the electrode 300. According to some examples, as described above in relation to the electrode 250, the steering mechanism of the handle 204 is further configured to proximally retract the electrode 300.

According to some examples, the distal portion 302 of the electrode 300 comprises at least one strut 310. Although the electrode 300 is illustrated and described herein as comprising a single strut 310, however this is not meant to be limiting in any way, and additional struts 310 may be provided.

According to some examples, strut 310 extends from a first end 311 to a second end 312 and further comprises: a forward section 313; a rear section 314; an inner face 315; and an outer face 316, outer face 316 opposing inner face 315. The term “forward section”, as used herein, means a section at the end of the electrode 300. The term “rear section”, as used herein, means a section at the beginning of the distal portion 302, near center portion 303, such that the rear section 314 extends between the center portion 303 and the forward section 313. According to some examples, when electrode 300 is in a contained state within the nosecone 220, the strut 310 is generally straight and the forward section 313 is distal to the rear section 314.

According to some examples, when the distal portion 302 of the electrode 300 is in a free state, at least the forward section 313 of the strut 310 is generally curved. According to some examples, the inner face 315 of the forward section 313 comprises a generally convex shape and the outer face 316 of the forward section 313 comprises a generally concave shape. According to some examples, the generally curved forward section 313 defines at least a portion of an ellipse. According to some examples, the generally curved forward section 313 defines at least half of an ellipse. According to some examples, the generally curved forward section 313 defines more than half of an ellipse. According to some examples, the generally curved forward section 313 defines at least a portion of a circle. According to some examples, the generally curved forward section 313 defines at least half of a circle. According to some examples, the generally curved forward section 313 defines more than half of a circle.

According to some examples, when the distal portion 302 of the electrode 300 is the free state, the rear section 314 is generally straight. According to some examples, the forward section 313 extends from the rear section 314. According to some examples, the forward section 313 is an extension of the rear section 314. Thus, in such examples, no additional parts, or gaps, are present between the forward section 313 and the rear section 314. Although electrode 300 is illustrated and described herein in such an example where the rear section 314 is generally straight, this is not meant to be limiting in any way, and the entirety of the distal portion 302 of the electrode 300 can be generally curved when in the free state.

According to some examples, as described above, the delivery apparatus 202 comprises an electrode 320. FIG. 8A shows the electrode 320 in a contained state and FIG. 8B shows the electrode 320 in a free state. According to some examples, the electrode 320 is in all respects similar to electrode 250 described above, with the exception that the electrode 320 comprises two struts 260 that extend into the front section 290.

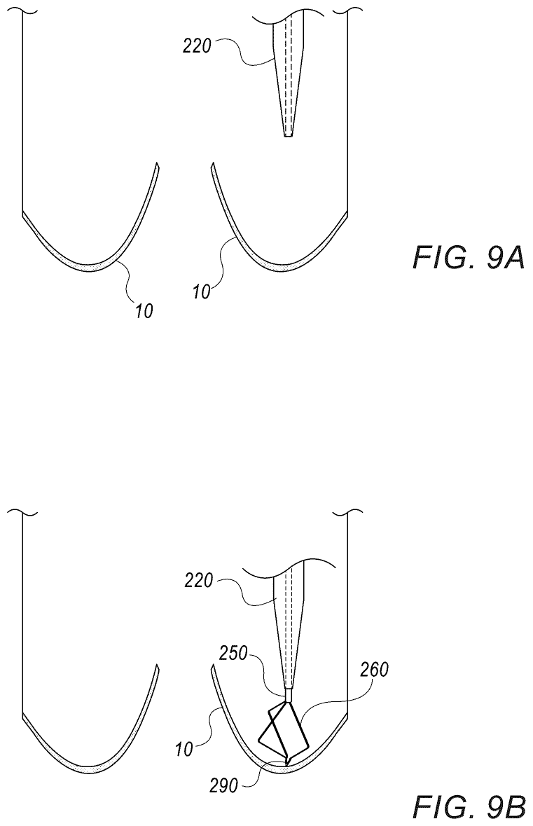

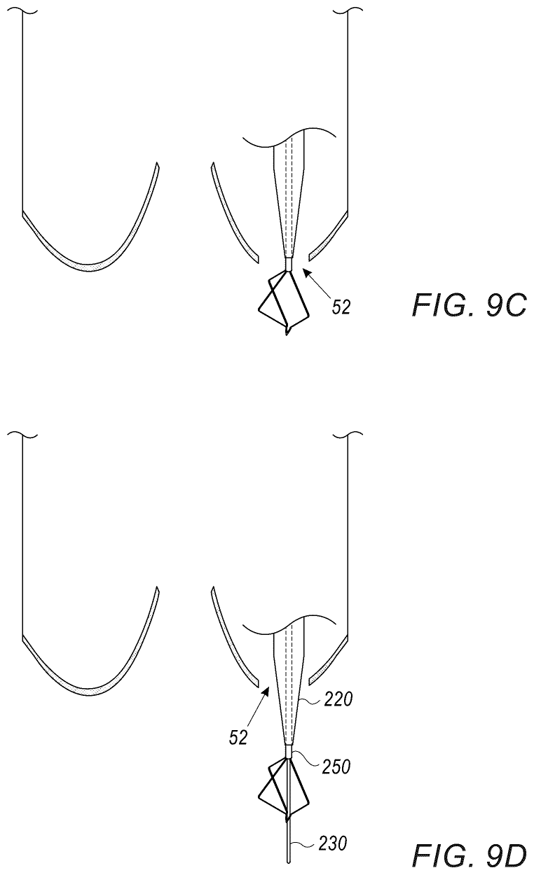

FIGS. 9A-9D illustrate various steps of an example of a perforation method. In a first step, as illustrated in FIG. 9A, a delivery apparatus, optionally the delivery apparatus 202 of perforation assembly 200 is advanced to the vicinity of a predetermined anatomical location. For simplicity, only the nosecone 220 of the delivery apparatus 202 is shown, however this is not meant to be limiting in any way. Furthermore, as described above, in some examples a nosecone 220 is not provided. In the illustrated example of FIG. 9A, the predetermined anatomical location is in the ascending aorta, however this is not meant to be limiting in any way.

It is to be understood that the perforation assembly 200 may be configured to form a tissue opening through other tissues in a patient's body. For example, prosthetic devices can be delivered to the left atrium or the left ventricle in a transseptal approach, wherein a delivery apparatus is passed through the vena cava, into the right atrium, and through the interatrial septum tissue. Such delivery approaches require puncturing the interatrial septum. Thus, in some examples, a perforation assembly 200 may be utilized to form an opening through the interatrial septum, for example at the site of the fossa ovalis, which is a region of the septum containing tissue of lesser thickness than is typical of the rest of the septum.

In some examples, the guidewire 230 (shown in FIG. 9D) can be used to advance delivery apparatus 202 towards the target tissue, such as a host valvular structure 12. For examples, when advanced toward the aortic annulus, the guidewire 230 can be first advanced toward the native heart valve and optionally pass between the leaflets, extending to some extent into the left ventricle, allowing advancement of the delivery apparatus 202 thereover toward the native annulus. Once the nosecone 220 is positioned in the vicinity of the native aortic valve, the guidewire 230 can be retracted back into the nosecone 220 and/or nosecone shaft 203, optionally concealing the distal portion 231 of guidewire 230, partially or completely, within nosecone 220 and/or nosecone shaft 203. The distal portion of the delivery apparatus 202 can be then steered toward one of the host leaflets 10, for example by a steering mechanism applied to outer delivery shaft 208, positioning the nosecone 220 proximate the host leaflet 10 (see FIG. 9A), at which point the electrode 250 can be axially translated in a distal direction to expose the distal portion thereof, allowing it to assume the non-straight configuration, as shown for example in FIG. 9B.

Although FIGS. 9A-9D are illustrated in relation to electrode 250, this is not meant to be limiting in any way, and the same method can be used in relation to electrodes 300 and 320.

As described above, an electric current is provided to the electrode 250, and the provided energy cuts a predetermined perforation in a section of material at the predetermined anatomical location. According to some examples, the material can be tissue (for example, a leaflet 30 of the native heart valve) or a portion of an implanted artificial structure (for example, a leaflet 114 of a previously implanted prosthetic valve). As described above, according to some examples the provided electric current generates RF energy that when applied to the material creates the perforation.

According to some examples, as described above, the electrode 250 comprises a front section 290. In such examples, an initial perforation can be created by the electric current flowing through the front section 290, followed by the struts of the electrode 250, thereby creating an opening 52, such as a leaflet opening 52, as shown in FIG. 9C.

According to some examples, as described above, the electric current can be controlled to be generated only when the electrode 250 is present at the desired location. Thus, accidental perforation of other tissue can be avoided. As further described above, an electrically conductive surface of a guidewire can be provided and used as well for forming the initial portion of the opening 52.

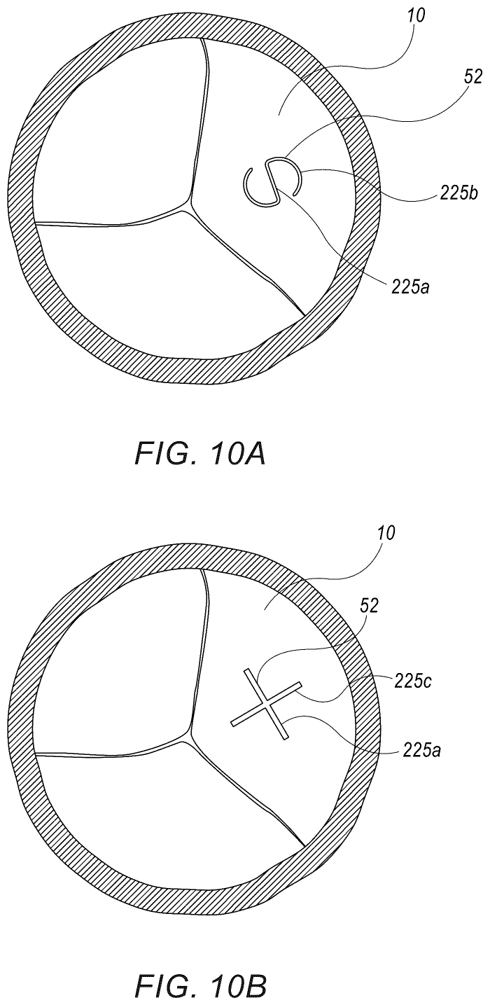

According to some examples, opening 52 is created by a plurality of perforations formed by rotating the electrode in a predetermined configuration. For examples, in regards to electrode 320 (shown in FIGS. 8A-8B), an initial elongated perforation 225a can be created and then electrode 320 can be rotated in either direction to create a pair of curved perforations 225b, as shown in FIG. 10A. The combination of elongated perforation 225a and curved perforations 225b form opening 52. According to some examples, the electrode 320 can be axially translated in the proximal direction, to be proximal to the leaflet 10, before being rotated to create curved perforations 225b. According to some examples, the electrode 320 can be axially translated in the distal direction, to be distal to the leaflet 10, before being rotated to create curved perforations 225b. According to some examples, the electrode 320 can rotated to create curved perforations 225b while being positioned within perforation 225a.

In another example, shown in FIG. 10B, in relation to the electrode 320, an initial elongated perforation 225a can be created and then electrode 320 can be rotated by approximately 90 degrees to create an additional elongated perforation 225c, thus creating a pair of elongated perforation in a ‘plus’ configuration. The combination of perforations 225a and 225c form opening 52.

Although FIGS. 10A-10B illustrate perforations created by electrode 320, this is not meant to be limiting in any way, and various respective perforations can be created by electrodes 250 and 300 as desired. Similarly, other perforations, at different angles, can be created by electrode 320.

According to some examples, such configuration of the struts of the electrodes described herein can provide a large opening with only a few perforations. Nevertheless, it is to be understood that any other rotation angle is contemplated, and that the procedure can be repeated any desired number of times to create multiple intersecting elongated perforations, such as in the shape of an asterisk or any other shape. If a single elongated shaped perforation 225 is formed, the leaflet opening 52 is defined thereby. If several elongated shaped perforations 225 are formed, they form together the leaflet opening 52, as described above.

In some examples, after creating the opening 52, the guidewire 230 can be axially translated in the distal direction to pass through the host leaflet 10

In some examples, for example in relation to electrode 300 (shown in FIGS. 7A-7B) the extent to which the forward section 313 of the electrode 300 is exposed from nosecone 220, dictates the projected length of the forward section 313 on host leaflet 10, thus controlling the desired length of the perforation. For example, the forward section 313 can only partially extend past nosecone 220, such that only a portion of the forward section 313 is exposed, while the remainder is retained within the nosecone 220. Advancement of the limited exposed portion of the forward section 313 through the host leaflet 10 can serve to form a relatively short elongated shaped perforation. In contrast, exposing a larger portion of the forward section 313, such as optionally the full length of the front sections 313 extending out of the nosecone 220, can serve to form a longer elongated shaped perforation. This maneuverability allows the practitioner to select the desired length of an elongated shaped perforation formed by the electrode 300 according to the target tissue characteristics, such as the type of tissue and/or patient-specific leaflet size and other anatomical characteristics.

According to some examples, as illustrated in FIG. 9D, the guidewire 230 is distally advanced through the opening 52, optionally followed by the nosecone 220, as will be further described below. According to some examples, nosecone 220 comprises a tapering distal portion, that can taper from the distal end to a larger diameter in the proximal direction. If the nosecone 220 tapers to a maximal diameter which is greater than the length of the created perforations formed by the electrode, advancement of the nosecone 220 through the leaflet opening 52 can serve to further dilate the leaflet opening 52.

According to some examples, as will be described further below, the distal portion of the electrode can be retracted back into the nosecone 220. For example, in relation to electrodes 250 and 320, the shape of the struts 260 causes struts 260 to collapse when retracted into the nosecone 220 and return to their original shape. Similarly, in relation to electrode 300, the shape of the strut 310 causes the strut 310 to be straightened out when retracted into the nosccone 220.

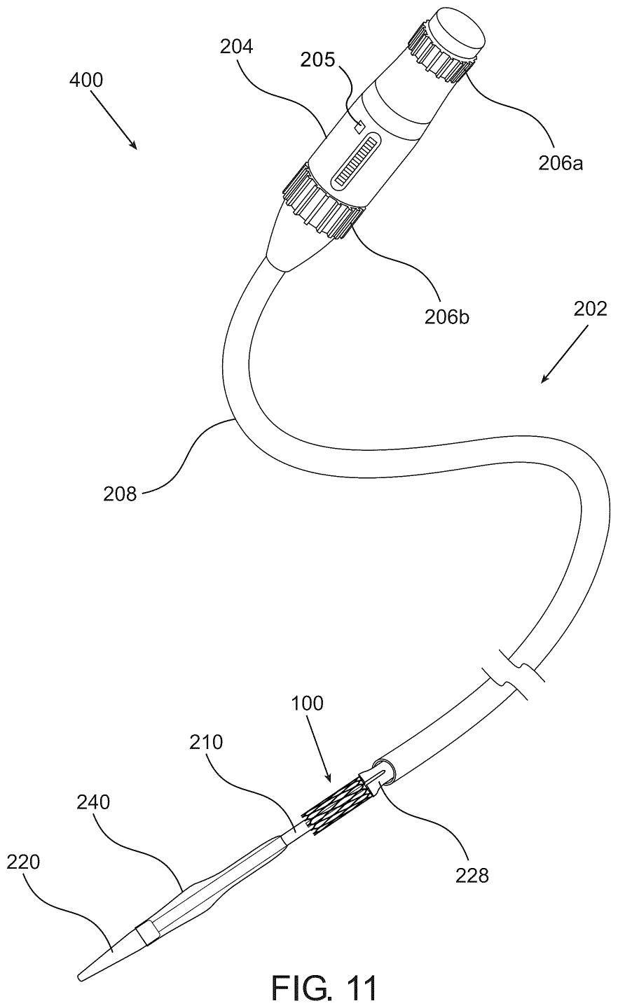

FIG. 11 shows a perspective view of an exemplary delivery assembly 400. According to some examples, delivery assembly 400 is in all respects similar to perforation assembly 200 (shown in FIG. 4), with the exception that a balloon expandable prosthetic valve 100 is provided, with an inflatable balloon 240, mounted on a distal portion of a balloon catheter 210.

The balloon expandable prosthetic valve 100 can be carried in a crimped state over the balloon catheter 210. According to some examples, the outer delivery shaft 208 can concentrically extend over the balloon catheter 210. In some examples, a push shaft 228 can be further provided, the push shaft 228 disposed over the balloon catheter 210. According to some examples, the push shaft 228 is provided between the balloon catheter 210 and the outer shaft 208.

According to some examples, the outer delivery shaft 208, the push shaft 228 and the balloon catheter 210 can be configured to be axially movable relative to each other. For example, a proximally oriented movement of the outer delivery shaft 208 relative to the balloon catheter 210, or a distally oriented movement of the balloon catheter 210 relative to the outer delivery shaft 208, can expose the prosthetic valve 100 from the outer delivery shaft 208. As described above, in some examples, the delivery apparatus 202 can further include a nosecone 220 carried by a nosecone shaft 203 (hidden from view in FIG. 11, but shown in FIG. 4) extending through a lumen of the balloon catheter 210 (not shown).

In some examples, the delivery assembly 400 can be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

According to some examples, nosecone shaft 203, balloon catheter 210, and optional outer delivery shaft 208, can be formed from any of various suitable materials, such as nylon, braided stainless steel wires, or a polyether block amide (commercially available as Pebax®). In some examples, nosecone shaft 203, balloon catheter 210, and optional outer delivery shaft 208, have longitudinal sections formed from different materials in order to vary the flexibility of the shafts along their lengths. In some examples, nosecone shaft 203 comprises an inner liner or layer formed of Teflon® to minimize sliding friction with guidewire 230.

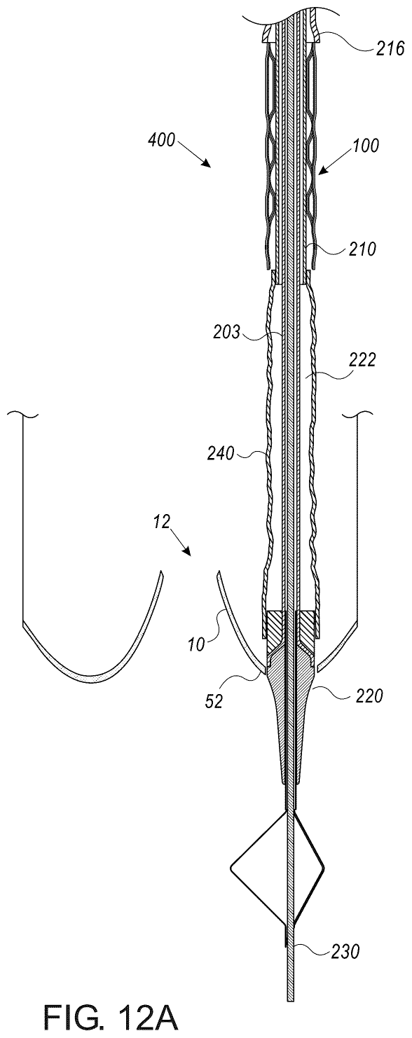

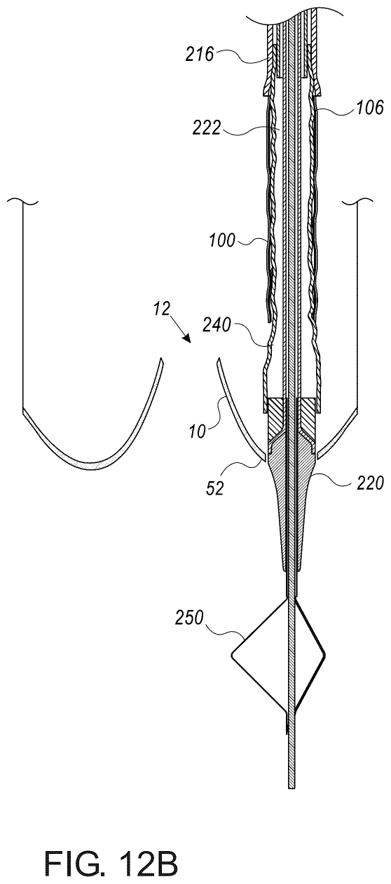

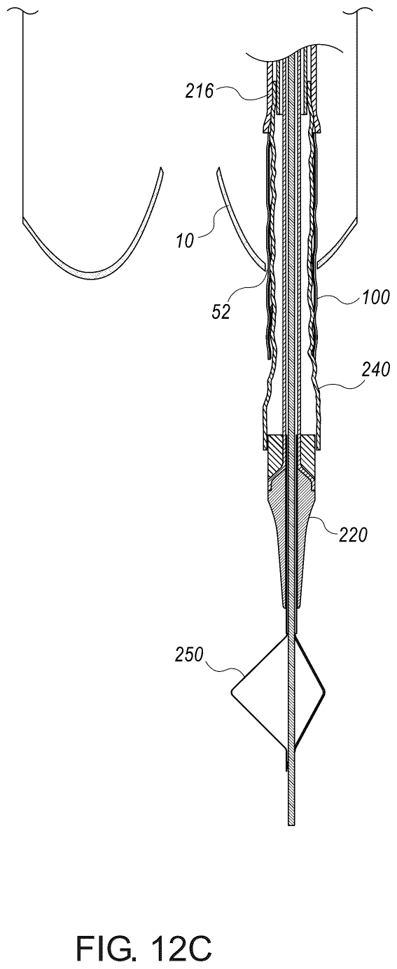

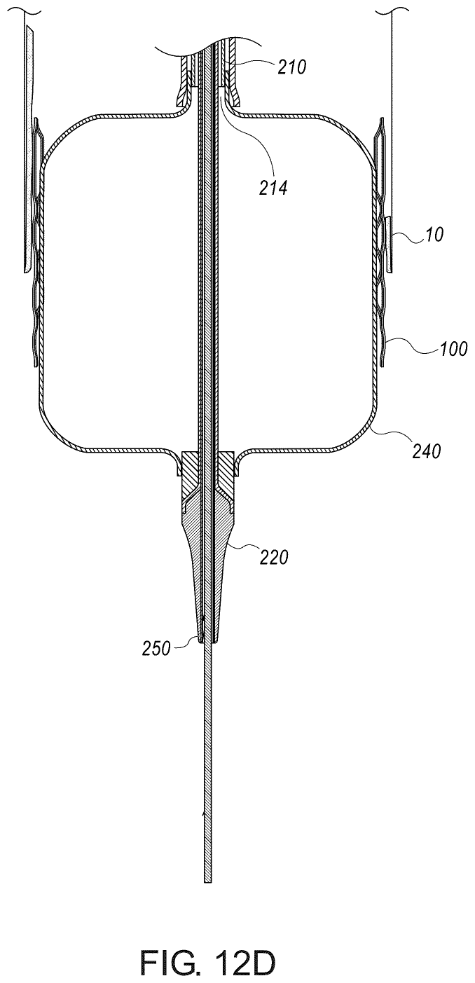

According to some examples, the nosecone shaft 203 is sized such that an annular space is formed within the balloon catheter lumen between the balloon catheter 210 and the nosecone shaft 203 along the length of the balloon catheter 210. This annular space is in fluid communication with one or more balloon catheter openings 214 (shown in FIG. 12D) exposed to an internal cavity 222 of the balloon 240, which can be in fluid communication with a fluid source (for example, a syringe or a pump) that can inject an inflation fluid (for example, saline) into the balloon cavity 222. In this way, fluid from the fluid source can flow through the balloon catheter lumen, and into balloon cavity 222, which serves to inflate the balloon 240 and expand and deploy a prosthetic valve 100 disposed thereon. The pressure of the inflation fluid within balloon cavity 222 may provide the force that allows the balloon 240 to expand a prosthetic valve 100 disposed thereon. Further, the balloon catheter lumen may be configured to withdraw fluid from balloon cavity 222, to deflate the balloon 240.

While the balloon catheter 210 is shown to terminate at a proximal end of the balloon 240 in in some examples, the balloon catheter 210 can extend farther in the distal direction (examples not illustrated), through a portion or through the entire length of the balloon cavity 222, and one or more of the balloon catheter opening(s) 214 can be formed on the sidewall of the balloon catheter 210, exposed laterally to the balloon cavity 222.

According to some examples, the proximal ends of the catheter 210, the outer delivery shaft 208, the push shaft 228, and optionally the nosecone shaft 203, are coupled to the handle 204. According to some examples, during delivery of the prosthetic valve 100 the handle 204 is maneuvered by an operator (for example, a clinician or a surgeon) to axially advance or retract components of the delivery apparatus 202, such as the nosecone shaft 203, the catheter 210, the outer delivery shaft 208, the push shaft 228, and perforating member 230 (for example guidewire 230), through the patient's vasculature and/or along the target site of implantation, as well as to inflate the balloon 240 mounted on the catheter 210, so as to expand the prosthetic valve 100, and to deflate the balloon 240 and retract the delivery apparatus 202 once the prosthetic valve 100 is mounted in the implantation site (for example, within the host valve).

According to some examples, the prosthetic valve 100 is carried by the delivery apparatus 202 during delivery in a crimped state, and expanded by balloon inflation to secure it in a native heart valve annulus (such as aortic annulus 24) or against a previously implanted prosthetic valve. In an exemplary implantation procedure, the prosthetic valve 100 is initially crimped over the catheter 210, proximal to the inflatable balloon 240. Because prosthetic valve 100 is crimped at a location different from the location of balloon 240, prosthetic valve 100 can be crimped to a lower profile than would be possible if it was crimped on top of balloon 240. This lower profile permits the clinician to more easily navigate the perforation assembly 200 (including crimped prosthetic valve 100) through a patient's vasculature to the treatment location. The lower profile of the crimped prosthetic valve is in some examples helpful when navigating through portions of the patient's vasculature which are particularly narrow, such as the iliac artery.

According to some examples, the balloon 240 is secured to balloon catheter 210 at the balloon's proximal end, and to either the balloon catheter 210, the nosecone shaft 203 or the nosecone 220 at its distal end. The distal end portion of the push shaft 228 is positioned proximal to the outflow end of the prosthetic valve 100.

According to some examples, the balloon catheter 210 extends through the handle 204 and is fluidly connectable to a fluid source for inflating the balloon 240. The fluid source comprises an inflation fluid. The term “inflation fluid”, as used herein, means a fluid (for example, saline, though other liquids or gas can be used) used for inflating the balloon 240. An inflation fluid source is in fluid communication with the balloon catheter lumen, such as the annular space between the inner face of balloon catheter 210 and the outer face of nosecone shaft 203 extending therethrough, such that fluid from the fluid source can flow through the balloon catheter lumen, and into the balloon 240 to inflate it.

When reaching the site of implantation, the delivery assembly 202 can be utilized to modify at least one host leaflet 10, as described above in relation to FIGS. 9A-9D, after which the deflated balloon, carrying crimped valve 100 thereover, can be advanced to the target site to expand the prosthetic valve.

Prior to balloon 240 inflation, the push shaft 228 can be advanced distally, allowing its distal end portion to contact and push against the outflow end of prosthetic valve 100, pushing the valve 100 distally therewith. The distal end of push shaft 228 is dimensioned to engage with the outflow end of prosthetic valve 100 in a crimped configuration of the valve. In some examples, the distal end portion of the push shaft 228 can be flared radially outward, to terminate at a wider-diameter that can contact the prosthetic valve 100 in its crimped state. Push shaft 228 can then be advanced distally, pushing the prosthetic valve 100 therewith, until the crimped prosthetic valve 100 is disposed around the balloon 240, at which point the balloon 240 can be inflated to radially expand the prosthetic valve 100. Once the prosthetic valve 100 is expanded to its functional diameter within a native annulus or within a previously implanted prosthetic host valve, the balloon 240 can be deflated, and the delivery apparatus 202 can be retrieved from the patient's body.

In some examples, any exemplary delivery assembly of the current disclosure can be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

Although delivery assembly 400 is described herein as carrying a prosthetic valve, this is not meant to be limiting in any way and the perforation assembly 200 and delivery assembly 400 can be used for implantation of other prosthetic devices aside from prosthetic valves, such as stents or grafts.

FIGS. 12A-12D show subsequent steps of a method utilizing delivery assembly 400, following steps equivalent to those described above and illustrated in FIGS. 9A-9D. Delivery assembly 400 is illustrated as comprising electrode 250, however this is not meant to be limiting in any way, and any suitable electrode, such as electrode 300 or electrode 320, can be utilized. After formation of the leaflet opening 52, and optional further enlargement of the leaflet opening 52 by the nosecone 220 as shown in FIGS. 9D and 12A, the push shaft 216 can be utilized to distally advance the crimped prosthetic valve 100 toward and around balloon 240, as shown in FIG. 12B.

According to some examples, the deflated balloon 240 and the prosthetic valve 100 disposed thereover can be then advanced and positioned within the leaflet opening 52, as shown in FIG. 12C. The push shaft 216 can remain in position, abutting the outflow end 106 of the prosthetic valve 100 during advancement into and through the leaflet opening 52, to provide a counter force that resists proximal displacement of the prosthetic valve 100 during insertion into the leaflet opening 52.

At this stage, delivering inflation fluid into the balloon cavity 222 allows the balloon 240 to inflate and expand the prosthetic valve 100, as shown in FIG. 12D. As described in more detail below, expanding the guest prosthetic valve 100 to the radially expanded configuration within the leaflet opening 52 of the host leaflet 10 may facilitate preserving access to the coronary arteries 34, 36 and/or maintaining sufficient perfusion of blood to the coronary arteries 34, 36 through the frame 102 of the guest prosthetic valve 100.

While the prosthetic valve 100 is shown in FIG. 12B to be pushed distally to the position of balloon 240 after forming the leaflet opening 52, it is to be understood that the prosthetic valve 100 can be pushed by push shaft 216 over balloon 240 at any other stage prior to advancement of the balloon 240 into the leaflet opening 52 as shown in FIG. 12C. For example, the prosthetic valve 100 can be pushed over balloon 240 upon approximation to the site of implantation, such as upon reaching the region of the ascending aorta 26 even prior to forming the perforation described above in relation to FIGS. 9A-9D. Similarly, the prosthetic valve 100 can be pushed distally after the perforation is performed, but before the nosecone 220 is advanced through the leaflet 10. Similarly, it is to be understood that in some examples, the delivery apparatus can be provided without a push shaft 216, and/or that the prosthetic valve 100 can be crimped around the outer balloon 240 and delivered through the patient's vasculature in this position.

With the guest prosthetic valve 100 received within the leaflet opening 52, radially expanding the guest prosthetic valve, as shown in FIG. 12D, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell opening 112 of the guest prosthetic valve 100.

According to some examples, as described above, the electrode 250 (or electrodes 300/320) can be proximally retracted into the nosecone 220. According to some examples, the electrode 250 is retracted before advancing the nosecone 220 through the leaflet opening 52.

As mentioned, the delivery assemblies and methods of the current specification can be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures.

Although the above has been described in relation to an electrode (250, 300 or 320), separate from a guidewire 230, this is not meant to be limiting in any way. According to some examples (not shown) a guidewire comprising an electrically conductive surface and being shaped as one of electrodes 250, 300 or 320 can be provided.

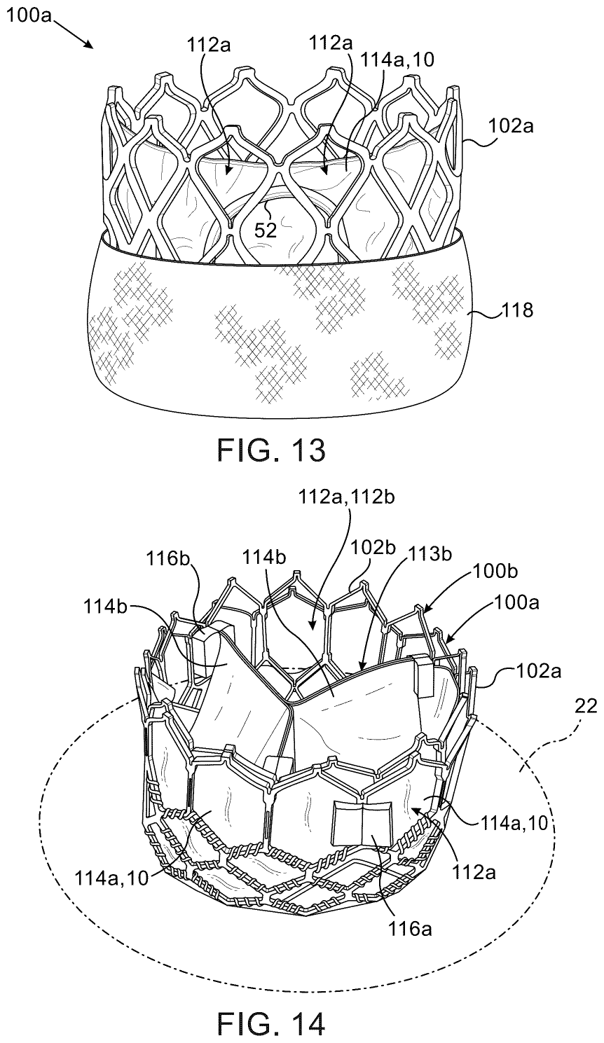

FIG. 13 shows a previously implanted prosthetic valve 100a subsequent to forming the leaflet opening 52, for example subsequent to the method described above with respect to FIGS. 9A-9D. FIG. 14 shows a configuration in which a second prosthetic valve 100b comprises been expanded within the leaflet opening 52 of a host prosthetic valve 100a. In the example of FIG. 14, the guest prosthetic valve 100b is the same type of valve as the host prosthetic valve 100a. It is to be understood, however, that the methods described herein, when implemented in ViV procedures, also may be applied to any other suitable valvular structures, such as different prosthetic valves and/or native heart valves. For example, the guest prosthetic valve 100b need not be the same type of valve as the host prosthetic valve 100a.

In the example of FIG. 13, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 14, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

While the methods disclosed herein refer to forming a leaflet opening 52 in a host leaflet 10, prior to positioning and expanding a prosthetic valve 100, it is to be understood that any of the methods can comprise, in some examples, repeating one or more steps disclosed throughout the current specification to form a plurality of punctures and openings in the host valvular structure 12. For example, steps described above with respect to FIGS. 6A-6D can be performed for forming a first leaflet opening in a first host leaflet, after which the delivery apparatus can be retracted from the first host leaflet and steered toward another host leaflet, after which the same steps can be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be beneficial in examples in which the frame of a host prosthetic valve extends axially in a downstream direction beyond one or both of the coronary arteries when the guest prosthetic valve is implanted within a native heart valve.

Specifically, in some patient anatomies, the left coronary artery is positioned lower (that is, proximate to the host valvular structure) than the right coronary artery. In such examples, the right coronary artery may be sufficiently far from the host valvular structure that implanting the guest prosthetic heart valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In other examples, however, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic heart valve may ensure the ability for future access into both coronary arteries or perfusion through the frame to both coronary arteries during the diastole phase of the cardiac cycle. As a more specific example, the host valvular structure can be modified such that the guest prosthetic valve is implanted by being expanded in a leaflet opening of a first host leaflet that faces the left coronary artery, and such that the second leaflet opening is formed in a second host leaflet that faces the right coronary artery (or vice-versa).

In some examples, forming the first leaflet opening can be performed prior to forming the second leaflet opening. In other examples, forming the second leaflet opening can be performed prior to forming the first leaflet opening. In some examples, the order of forming leaflet openings is chosen such that the final leaflet opening is formed in the host leaflet in which the guest prosthetic valve 100 is to be positioned and expanded, such as over a balloon as described above with respect to FIGS. 12A-12D.

It is to be understood that the guest prosthetic valve 100 is not limited to being implanted within an opening 52 of a leaflet. For example, in cases where the electrode 250, and optionally the nosecone 220, forms a full tear in a host leaflet that extends to the coaptation edge of the leaflet, the guest prosthetic valve 100 can be positioned at a location between the leaflets of the host valvular structure, for example by retracting the delivery apparatus from the host leaflet in which a leaflet opening is formed, repositioning and readvancing it such that the deflated valve expansion balloon, along with the prosthetic valve 100 disposed thereon, is positioned between the host leaflets, and then inflating the valve expansion balloon to expand the prosthetic valve 100. In some examples, such as in cases where the opening 52 does not form a full tear in the leaflet, the guest prosthetic heart valve can be positioned at a location between the leaflets of the host valvular structure 12 (such that the perforation assembly 200 used to implant to guest prosthetic valve 100 does not extend through the leaflet opening 52) and then expanded. In such cases, the opening 52 may provide sufficient open space through which blood may flow into the coronary ostia, and/or through which additional access devices, such as coronary catheters, can pass during future interventional procedures.

Any of the assemblies, devices, apparatuses, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A perforation assembly, comprising: a delivery apparatus; and an electrode extending from a proximal portion to a distal portion thereof, through the delivery apparatus, the distal portion of the electrode configured to extend distally from the delivery apparatus, wherein the distal portion of the electrode comprises a non-straight shape in a free state thereof.

Example 2. The assembly of any example herein, particularly example 1, wherein the distal portion of the electrode comprises at least one strut.

Example 3. The assembly of any example herein, particularly example 2, wherein, in the free state of the distal portion of the electrode, each of the at least one strut comprises a rear section, a predetermined forward angle being defined between the rear section and a longitudinal axis of the electrode.

Example 4. The assembly of any example herein, particularly example 3, wherein the forward angle is less than, or equal to, 90 degrees.

Example 5. The assembly of any example herein, particularly example 2, wherein, in the free state of the distal portion of the electrode, each of the at least one strut comprises: a rear section extending from a proximal end to a distal end thereof; and a forward section extending from a proximal end to a distal end thereof, wherein a predetermined center angle is defined between the rear section and the forward section, the predetermined center angle being a non-zero angle and a non-straight angle.

Example 6. The assembly of any example herein, particularly example 5, wherein, for each of the at least one strut, the rear section extends away from a longitudinal axis of the electrode and the forward section extending towards the longitudinal axis of the electrode.

Example 7. The assembly of any example herein, particularly example 6, wherein, for each of the at least one strut, the forward section of the strut is an extension of the rear section of the strut.

Example 8. The assembly of any example herein, particularly any one of examples 5-7, wherein, for each of the at least one strut, the predetermined center angle is less than 180 degrees.

Example 9. The assembly of any example herein, particularly any one of examples 5-8, wherein, for each of the at least one strut, the distal end of the rear section of the strut is generally curved and the proximal end of the forward section of the strut is generally curved.

Example 10. The assembly of any example herein, particularly example 9, wherein, for each of the at least one strut, the distal end of the forward section of the strut and the proximal end of the forward section of the strut define a center section of the strut, an outer face of the center section of the strut comprising a generally convex shape and an inner face of the center section of the strut comprising a generally concave shape, wherein the inner face of the center section of the strut faces the longitudinal axis of the electrode.

Example 11. The assembly of any example herein, particularly any one of examples 5-10, wherein, for each of the at least one strut, the proximal end of the rear section of the strut is generally curved.

Example 12. The assembly of any example herein, particularly any one of examples 5-11, wherein, for each of the at least one strut, an inner face of the proximal end of the rear section of the strut comprises a generally convex shape and an outer face of the proximal end of the rear section of the strut comprises a generally concave shape, wherein the inner face of the proximal end of the rear section of the strut faces the longitudinal axis of the electrode.

Example 13. The assembly of any example herein, particularly any one of examples 5-12, wherein, for each of the at least one strut, the distal end of the forward section of the strut is generally curved.

Example 14. The assembly of any example herein, particularly any one of examples 5-13, wherein, for each of the at least one strut, an inner face of the distal end of the rear section of the strut comprises a generally convex shape and an outer face of the proximal end of the rear section of the strut comprises a generally concave shape, wherein the inner face of the proximal end of the rear section of the strut faces the longitudinal axis of the electrode.

Example 15. The assembly of any example herein, particularly any one of examples 5-14, wherein the distal portion of the electrode further comprises a front section extending from the distal end of the forward section of each of the at least one strut.

Example 16. The assembly of any example herein, particularly example 15, wherein the front section of the distal portion of the electrode extends from a proximal end to a distal end thereof, the distal end of the front section forming a tip.

Example 17. The assembly of any example herein, particularly example 16, wherein the tip of the front section of the distal portion of the electrode defines a generally triangle shape.

Example 18. The assembly of any example herein, particularly any one of examples 15-17, wherein the front section of the distal portion of the electrode comprises a plurality of arms.

Example 19. The assembly of any example herein, particularly example 18, wherein each of the plurality of arms of the front section of the distal portion of the electrode extends from the distal end of the forward section of a respective one of the at least one strut of the electrode.

Example 20. The assembly of any example herein, particularly any one of examples 2-19, wherein, in a contained state of the distal portion of the electrode, each of the at least one strut is generally straight. 21. The assembly of any one of examples 2-20, wherein the electrode further comprises a center portion extending between the proximal portion and the distal portion, a cross-section of the center portion comprising a generally closed shape.

Example 22. The assembly of any example herein, particularly example 21, wherein the center portion of the electrode is generally tubular shaped.

Example 23. The assembly of any example herein, particularly example 21 or 22, wherein each of the at least one strut is attached to the center portion of the electrode.

Example 24. The assembly of any example herein, particularly example 2, wherein, in the free state of the distal portion of the electrode, a rear section of the at least one strut is generally curved.

Example 25. The assembly of any example herein, particularly example 24, wherein an outer face of the generally curved rear section of the at least one strut comprises a generally concave shape and an inner face of the generally curved rear section of the at least one strut comprises a generally convex shape.

Example 26. The assembly of any example herein, particularly example 24 or 25, wherein the generally curved rear section of the at least one strut defines at least a portion of an ellipse.

Example 27. The assembly of any example herein, particularly example 26, wherein the portion of the ellipse is at least half of the ellipse.

Example 28. The assembly of any example herein, particularly example 27, wherein the portion of the ellipse is greater than half of the ellipse.

Example 29. The assembly of any example herein, particularly any one of examples 26-28, wherein the ellipse is a circle.

Example 30. The assembly of any example herein, particularly any one of examples 24-29, wherein, in the free state of the distal portion of the electrode, the at least one strut of distal portion of the electrode further comprises a forward section, the forward section being generally straight.

Example 31. The assembly of any example herein, particularly example 30, wherein the rear section of the at least one strut extends from the forward section of the distal portion of the at least one strut.

Example 32. The assembly of any example herein, particularly example 31, wherein the electrode further comprises a center portion extending between the proximal portion and the distal portion thereof, a cross-section of the center portion comprising a generally closed shape.

Example 33. The assembly of any example herein, particularly example 32, wherein the center portion of the electrode is generally tubular shaped.

Example 34. The assembly of any example herein, particularly example 32 or 33 wherein the forward section of the at least one strut extends between the center portion of the electrode and the rear section of the distal portion of the at least one strut.

Example 35. The assembly of any example herein, particularly example 34, wherein the forward section of the at least one strut is attached to the center portion of the electrode.

Example 36. The assembly of any example herein, particularly any one of examples 2-25, wherein the at least one strut comprises a plurality of struts.

Example 37. The assembly of any example herein, particularly example 36, wherein the plurality of struts comprises at least three struts.

Example 38. The assembly of any example herein, particularly example 37, wherein the plurality of struts consists of three struts.

Example 39. The assembly of any example herein, particularly example 37, wherein the plurality of struts consists of two struts.

Example 40. The assembly of any example herein, particularly any one of examples 21-23 and 32-35, wherein the delivery apparatus further comprises a delivery guidewire extending through the center portion of the electrode.

Example 41. The assembly of any example herein, particularly example 40, wherein the delivery apparatus comprises: a delivery nosecone comprising a nosecone channel, the electrode extending through the nosecone channel; and a nosecone shaft attached to the delivery nosecone and extending proximally therefrom, the delivery guidewire extending through a lumen of the nosecone shaft.

Example 42. The assembly of any example herein, particularly example 41, wherein the center portion of the electrode extends through the lumen of the nosecone shaft.

Example 43. The assembly of any example herein, particularly any one of examples 40-42, wherein a distal portion of the delivery guidewire comprises an electrically conductive surface.

Example 44. The assembly of any example herein, particularly example 43, further comprising an electric current source configured to provide a respective electric current to the guidewire.

Example 45. The assembly of any example herein, particularly example 44, wherein the provided electric current is an alternating current.

Example 46. The assembly of any example herein, particularly example 45, wherein a frequency of the alternating current is a radio-frequency.

Example 47. The assembly of any example herein, particularly any one of examples 44-46, wherein the electric current source is configured, responsive to a user input, to alternately provide the electric current and not provide the respective electric current.

Example 48. The assembly of any example herein, particularly any one of examples 44-47, wherein the electric current source is further configured to provide a respective electric current to the electrode.

Example 49. The assembly of any example herein, particularly any one of examples 40-48, further comprising an adjustment mechanism configured to distally advance the electrode.

Example 50. The assembly of any example herein, particularly example 49, wherein the adjustment mechanism is further arranged to distally advance the electrode.

Example 51. The assembly of any example herein, particularly example 50, wherein the adjustment mechanism is further arranged to rotate the electrode.

Example 52. The assembly of any example herein, particularly any one of examples 1-48, further comprising an adjustment mechanism configured to distally advance the electrode.

Example 53. The assembly of any example herein, particularly example 52, wherein the adjustment mechanism is further configured to rotate the electrode.

Example 54. The assembly of any example herein, particularly any one of examples 1-43 and 52-53, further comprising an electric current source configured to provide electric current to the electrode.

Example 55. The assembly of any example herein, particularly example 54, wherein the provided electric current is an alternating current.

Example 56. The assembly of any example herein, particularly example 55, wherein a frequency of the alternating current is a radio-frequency.

Example 57. The assembly of any example herein, particularly any one of examples 54-56, wherein the electric current source is configured, responsive to a user input, to alternately provide the electric current or not provide the electric current.

Example 58. The assembly of any example herein, particularly any one of examples 1-40, wherein the delivery apparatus comprises a delivery nosecone comprising a nosecone channel, the electrode extending through the nosecone channel.

Example 59. The assembly of any example herein, particularly example 58, wherein the distal portion of the electrode is pre-shaped such that responsive to the distal portion of the electrode being released from the nosecone channel the distal portion forms into the non-straight shape.

Example 60. The assembly of any example herein, particularly example 58 or 59, further comprising a prosthetic valve positioned proximally in relation to the delivery nosecone.

Example 61. The assembly of any example herein, particularly example 60, further comprising a push shaft configured to push the prosthetic valve distally towards the nosecone.

Example 62. The assembly of any example herein, particularly any one of examples 58-61, further comprising a balloon catheter extending to the nosecone and an inflatable balloon positioned over the balloon catheter.

Example 63. The assembly of any example herein, particularly any one of examples 1-62, wherein the electrode is monopolar.

Example 64. A perforation method, the method comprising: distally extending a distal portion of an electrode from a delivery apparatus to a predetermined anatomical location, the electrode comprising a non-straight shape in a free state thereof; and providing an electric current to the electrode such that the electric current creates a perforation in a section of material at the predetermined anatomical location.

Example 65. The method of any example herein, particularly example 64, wherein the perforation is created by radio-frequency energy generated by the provided electric current.

Example 66. The method of any example herein, particularly example 65, wherein the provided electric current is an alternating current.

Example 67. The method of any example herein, particularly example 66, wherein a frequency of the alternating current is a radio-frequency.

Example 68. The method of any example herein, particularly any one of examples 65-67, further comprising, responsive to a user input, alternately providing the electric current and not providing the electric current.

Example 69. The method of any example herein, particularly any one of examples 64-68, wherein the material at the predetermined anatomical location comprises tissue.

Example 70. The method of any example herein, particularly any one of examples 64-68, wherein the material at the predetermined anatomical location comprises a portion of an implanted artificial structure.

Example 71. The method of any example herein, particularly any one of examples 64-68, wherein the predetermined anatomical location is a native aortic valve.

Example 72. The method of any example herein, particularly example 71, wherein the material is a leaflet of the native aortic valve.

Example 73. The method of any example herein, particularly example 71, wherein the material is a portion of a previously implanted prosthetic valve.

Example 74. The method of any example herein, particularly any one of examples 64-73, further comprising rotating the distal portion of the electrode.

Example 75. The method of any example herein, particularly any one of examples 64-73, wherein subsequent to creating the perforation the distal portion of the electrode is rotated by a predetermined rotation angle, and an additional perforation is created by the rotated distal portion of the electrode.

Example 76. The method of any example herein, particularly example 75, wherein the predetermined rotation angle is about 90 degrees.

Example 77. The method of any example herein, particularly example 75 or 76, wherein the additional perforation is generally curved.

Example 78. The method of any example herein, particularly any one of examples 64-77, further comprising inserting an inflatable balloon through the perforation.

Example 79. The method of any example herein, particularly example 78, further comprising inflating the inflatable balloon.

Example 80. The method of any example herein, particularly any one of examples 64-79, further comprising inserting an expandable prosthetic valve into the perforation.

Example 81. The method of any example herein, particularly example 80, further comprising expanding the expandable prosthetic valve.

Example 82. The method of any example herein, particularly any one of examples 64-81, further comprising proximally retracting the electrode.

Example 83. The method of any example herein, particularly example 82, wherein the electrode is proximally retracted into a delivery nosecone.

Example 84. The method of any example herein, particularly example 83, wherein the proximal retraction of the electrode into the delivery nosecone straightens the non-straight shape.

Example 85. The method of any example herein, particularly any one of examples 64-84, wherein the distal portion of the electrode comprises at least one strut.

Example 86. The method of any example herein, particularly example 85, wherein, in the free state of the distal portion of the electrode, each of the at least one strut comprising a rear section, a predetermined forward angle being defined between the rear section and a longitudinal axis of the electrode.

Example 87. The method of any example herein, particularly example 86, wherein the forward angle is less than, or equal to, 90 degrees.

Example 88. The method of any example herein, particularly example 85, wherein, in the free state of the distal portion of the electrode, each of the at least one strut comprises: a rear section extending from a proximal end to a distal end thereof; and a forward section extending from a proximal end to a distal end thereof, wherein a predetermined center angle is defined between the rear section and the forward section, the predetermined center angle being a non-zero angle and a non-straight angle.

Example 89. The method of any example herein, particularly example 88, wherein, for each of the at least one strut, the rear section extends away from a longitudinal axis of the electrode and the forward section extending towards the longitudinal axis of the electrode.

Example 90. The method of any example herein, particularly example 89, wherein, for each of the at least one strut, the forward section of the strut is an extension of the rear section of the strut.

Example 91. The method of any example herein, particularly any one of examples 88-90, wherein, for each of the at least one strut, the predetermined center angle is less than 180 degrees.

Example 92. The method of any example herein, particularly any one of examples 88-91, wherein, for each of the at least one strut, the distal end of the rear section of the strut is generally curved and the proximal end of the forward section of the strut is generally curved.

Example 93. The method of any example herein, particularly example 92, wherein, for each of the at least one strut, the distal end of the forward section of the strut and the proximal end of the forward section of the strut define a center section of the strut, an outer face of the center section of the strut comprising a generally convex shape and an inner face of the center section of the strut comprising a generally concave shape, wherein the inner face of the center section of the strut faces the longitudinal axis of the electrode.

Example 94. The method of any example herein, particularly any one of examples 88-93, wherein, for each of the at least one strut, the proximal end of the rear section of the strut is generally curved.

Example 95. The method of any example herein, particularly any one of examples 88-94, wherein, for each of the at least one strut, an inner face of the proximal end of the rear section of the strut comprises a generally convex shape and an outer face of the proximal end of the rear section of the strut comprises a generally concave shape, wherein the inner face of the proximal end of the rear section of the strut faces the longitudinal axis of the electrode.

Example 96. The method of any example herein, particularly any one of examples 88-95, wherein, for each of the at least one strut, the distal end of the forward section of the strut is generally curved.

Example 97. The method of any example herein, particularly any one of examples 88-96, wherein, for each of the at least one strut, an inner face of the distal end of the rear section of the strut comprises a generally convex shape and an outer face of the proximal end of the rear section of the strut comprises a generally concave shape, wherein the inner face of the proximal end of the rear section of the strut faces the longitudinal axis of the electrode.

Example 98. The method of any example herein, particularly any one of examples 88-97, wherein the distal portion of the electrode further comprises a front section extending from the distal end of the forward section of each of the at least one strut.

Example 99. The method of e any example herein, particularly example 98, wherein the front section of the distal portion of the electrode extends from a proximal end to a distal end thereof, the distal end of the front section forming a tip.

Example 100. The method of any example herein, particularly example 99, wherein the tip of the front section of the distal portion of the electrode defines a generally triangle shape.

Example 101. The method of any example herein, particularly any one of examples 98-100, wherein the front section of the distal portion of the electrode comprises a plurality of arms.

Example 102. The method of any example herein, particularly example 101, wherein each of the plurality of arms of the front section of the distal portion of the electrode extends from the distal end of the forward section of a respective one of the at least one strut of the electrode.

Example 103. The method of any example herein, particularly any one of examples 88-102, wherein, in a contained state of the distal portion of the electrode, each of the at least one strut is generally straight.

Example 104. The method of any example herein, particularly any one of examples 88-103, wherein the electrode further comprises a center portion extending between a proximal portion of the electrode and the distal portion of the electrode, a cross-section of the center portion comprising a generally closed shape.

Example 105. The method of any example herein, particularly example 104, wherein the center portion of the electrode is generally tubular shaped.

Example 106. The method of any example herein, particularly example 104 or 105, wherein each of the at least one strut is attached to the center portion of the electrode.

Example 107. The method of any example herein, particularly example 88, wherein, in the free state of the distal portion of the electrode, a forward section of the at least one strut is generally curved.

Example 108. The method of any example herein, particularly example 107, wherein an outer face of the generally curved forward section of the at least one strut comprises a generally concave shape and an inner face of the generally curved forward section of the at least one strut comprises a generally convex shape.

Example 109. The method of any example herein, particularly example 107 or 108, wherein the generally curved forward section of the at least one strut defines at least a portion of an ellipse.

Example 110. The method of any example herein, particularly example 109, wherein the portion of the ellipse is at least half of the ellipse.

Example 111. The method of any example herein, particularly example 110, wherein the portion of the ellipse is greater than half of the ellipse.

Example 112. The method of any example herein, particularly any one of examples 109-111, wherein the ellipse is a circle.

Example 113. The method of any example herein, particularly any one of examples 107-112, wherein, in the free state of the distal portion of the electrode, the at least one strut of distal portion of the electrode further comprises a rear section, the rear section being generally straight.

Example 114. The method of any example herein, particularly example 113, wherein the forward section of the at least one strut extends from the rear section of the distal portion of the at least one strut.

Example 115. The method of any example herein, particularly example 114, wherein the electrode further comprises a center portion extending between a proximal portion of the electrode and the distal portion of the electrode, a cross-section of the center portion comprising a generally closed shape.

Example 116. The method of any example herein, particularly example 115, wherein the center portion of the electrode is generally tubular shaped.

Example 117. The method of any example herein, particularly example 115 or 116 wherein the rear section of the at least one strut extends between the center portion of the electrode and the forward section of the distal portion of the at least one strut.

Example 118. The method of any example herein, particularly example 117, wherein the rear section of the at least one strut is attached to the center portion of the electrode.

Example 119. The method of any example herein, particularly any one of examples 85-118, wherein the at least one strut comprises a plurality of struts.

Example 120. The method of any example herein, particularly example 119, wherein the plurality of struts comprises at least three struts.

Example 121. The method of any example herein, particularly example 120, wherein the plurality of struts consists of three struts.

Example 122. The method of any example herein, particularly example 120, wherein the plurality of struts consists of two struts.

Example 123. The method of any example herein, particularly any one of examples 64-122, further comprising distally advancing a delivery guidewire towards the predetermined anatomical location.

Example 124. The method of any example herein, particularly example 123, further comprising providing the electric current to the distally advanced delivery guidewire.

Example 125. The method of any example herein, particularly any one of examples 64-124, wherein the electrode is monopolar.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A perforation assembly, comprising:
a delivery apparatus; and
an electrode extending from a proximal portion to a distal portion thereof, through the delivery apparatus, the distal portion of the electrode configured to extend distally from the delivery apparatus,
wherein the distal portion of the electrode comprises a non-straight shape in a free state thereof.

2. The assembly of claim 1, wherein the distal portion of the electrode comprises at least one strut.

3. The assembly of claim 2, wherein, in the free state of the distal portion of the electrode, each of the at least one strut comprises a rear section, a predetermined forward angle being defined between the rear section and a longitudinal axis of the electrode, and wherein the forward angle is less than, or equal to, 90 degrees.

4. The assembly of claim 2, wherein, in the free state of the distal portion of the electrode, each of the at least one strut comprises:
a rear section extending from a proximal end to a distal end thereof; and
a forward section extending from a proximal end to a distal end thereof,
wherein a predetermined center angle is defined between the rear section and the forward section, the predetermined center angle being a non-zero angle and a non-straight angle, and
wherein, for each of the at least one strut, the rear section extends away from a longitudinal axis of the electrode and the forward section extending towards the longitudinal axis of the electrode.

5. The assembly of claim 4, wherein, for each of the at least one strut, the predetermined center angle is less than 180 degrees.

6. The assembly of claim 4, wherein, for each of the at least one strut, the distal end of the rear section of the strut is generally curved and the proximal end of the forward section of the strut is generally curved, the distal end of the forward section of the strut and the proximal end of the forward section of the strut defining a center section of the strut,
wherein an outer face of the center section of the strut comprises a generally convex shape and an inner face of the center section of the strut comprises a generally concave shape, and
wherein the inner face of the center section of the strut faces the longitudinal axis of the electrode.

7. The assembly of claim 4, wherein the distal portion of the electrode further comprises a front section extending from the distal end of the forward section of each of the at least one strut,
wherein the front section of the distal portion of the electrode extends from a proximal end to a distal end thereof, the distal end of the front section forming a tip; and
wherein the front section of the distal portion of the electrode comprises a plurality of arms.

8. The assembly of claim 2, wherein the at least one strut comprises a plurality of struts.

9. The assembly of claim 8, wherein the plurality of struts comprises at least three struts.

10. The assembly of claim 8, wherein the plurality of struts consists of two struts.

11. The assembly of claim 1, further comprising an electric current source configured to provide electric current to the electrode.

12. A perforation method, the method comprising:
distally extending a distal portion of an electrode from a delivery apparatus to a predetermined anatomical location, the electrode comprising a non-straight shape in a free state thereof; and
providing an electric current to the electrode such that the electric current creates a perforation in a section of material at the predetermined anatomical location.

13. The method of claim 12, wherein the material at the predetermined anatomical location comprises tissue.

14. The method of claim 12, wherein the material at the predetermined anatomical location comprises a portion of an implanted artificial structure.

15. The method of claim 12, wherein the predetermined anatomical location is a native aortic valve.

16. The method of claim 15, wherein the material is a leaflet of the native aortic valve.

17. The method of claim 15, wherein the material is a portion of a previously implanted prosthetic valve.

18. The method of claim 12, further comprising rotating the distal portion of the electrode.

19. The method of claim 12, wherein subsequent to creating the perforation the distal portion of the electrode is rotated by a predetermined rotation angle, and an additional perforation is created by the rotated distal portion of the electrode.

20. The method of claim 19, wherein the additional perforation is generally curved.
